SEQUENCE LISTING (12 sequences)

SEQ ID NO: 1 - DNA, 4100 bp, Artificial
  Synthetic Plasmid
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatatggtcttcacactcgaagatttcgttggggactggcgacagacagccggctacaacctggaccaagtcctcgagcagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgaattctcacggctttccgcctgaggttgaagagcaagccgccggtacattgcctatgtcctgcgcacaagaaagcggtatggaccggcacccagccgcttgtgcttcagctcgcatcaacgtctaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctggcctaactggcctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgggcctcggcggccaagctaggcaatccggtactgttggtaaagccaccatggtgagctaacgtagctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 4113 bp, Artificial
  Synthetic Plasmid
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgtttaaacgcggccgcggcctaactggcctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgggcctcggcggccaagctaggcaatccggtactgttggtaaagccaccatggtcttcacactcgaagatttcgttggggactggcgacagacagccggctacaacctggaccaagtcctcgagcagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgaattctcacggctttccgcctgaggttgaagagcaagccgccggtacattgcctatgtcctgcgcacaagaaagcggtatggaccggcacccagccgcttgtgcttcagctcgcatcaacgtctaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctggtaccgtttaaacctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 3 - DNA, 4027 bp, Artificial
  Synthetic Plasmid
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgtttaaacgcggccgcggcctaactggcctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgggcctcggccggctacaacctggaccaagtcctcgagcagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgaattctcacggctttccgcctgaggttgaagagcaagccgccggtacattgcctatgtcctgcgcacaagaaagcggtatggaccggcacccagccgcttgtgcttcagctcgcatcaacgtctaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctggtaccgtttaaacctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 4 - DNA, 4308 bp, Artificial
  Synthetic Plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctggcctaactggcctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgggcctcggcggccaagctaggcaatccggtactgttggtaaagccaccatggtcttcacactcgaagatttcgttggggactggcgacagacagccggctacaacctggaccaagtcctcgagcagggaggtgtgtccagtttgtttcagaatctcggggtctccgtaactccgatccaaaggattgtcctgagcggtgaaaatgggctgaagatcgatatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 73 bp, Artificial
  Synthetic Primer
tcgaggacttggtccaggttgtagccggctgtctgtcgccagtccccaacgaaatcttcgagtgtgaagacat

SEQ ID NO: 6 - DNA, 25 bp, Artificial
  Synthetic Primer
gccggctacaacctggaccaagtcc

SEQ ID NO: 7 - DNA, 101 bp, Artificial
  Synthetic Primer
agctttattgcggtagtttatcacagttaaattgctaacgcagtcagtgggcctcggcggccaagctaggcaatccggtactgttggtaaagccaccatgg

SEQ ID NO: 8 - DNA, 52 bp, Artificial
  Synthetic Primer
cgaggcccactgactgcgttagcaatttaactgtgataaactaccgcaataa

SEQ ID NO: 9 - DNA, 21 bp, Artificial
  Synthetic Primer
cgcaaatgggcggtaggcgtg

SEQ ID NO: 10 - DNA, 20 bp, Artificial
  Synthetic Primer
gtggtttgtccaaactcatc

SEQ ID NO: 11 - DNA, 23 bp, Artificial
  Synthetic Primer
cattctggcgtaaggccgcgact

SEQ ID NO: 12 - DNA, 27 bp, Artificial
  Synthetic Primer
ctagagtcgcggccttacgccagaatg

(19) United States
(12) Patent Application Publication
(10) Pub. No.: US 2022/0298587 A1
(43) Pub. Date: Sep. 22, 2022

(54) NOVEL CONSTRUCT

(71) Applicant: ARTIOS PHARMA LIMITED, Cambridge (GB)

(72) Inventors: Eeson RAJENDRA, Cambridge (GB); Helen ROBINSON, Cambridge (GB)

(73) Assignee: ARTIOS PHARMA LIMITED, Cambridge (GB)

(21) Appl. No.: 17/597,091

(22) PCT Filed: Jul. 1, 2020
(86) PCT No.: PCT/GB2020/051575

§ 371 Date: Dec. 26, 2021

(30) Foreign Application Priority Data
Jul. 1, 2019 (GB) 1909439.0

(51) Int. Cl. C12Q 1/6897; C12N 15/85

(52) U.S. Cl. CPC C12Q 1/6897; C12N 15/85; C12N 2800/10

(57) ABSTRACT

The invention relates to an NHEJ or MMEJ detection construct, to NHEJ or MMEJ detection assays comprising said construct and to a method of screening for an NHEJ or MMEJ modulator comprising said construct.

### FIELD OF THE INVENTION

The invention relates to an NHEJ or MMEJ detection construct, to NHEJ or MMEJ detection assays comprising said construct and to a method of screening for an NHEJ or MMEJ modulator comprising said construct.

### BACKGROUND OF THE INVENTION

Robust repair of DNA double-strand breaks (DSBs) is essential for the maintenance of genome stability and cell viability. In eukaryotic cells, DSBs can be repaired by one of three main pathways: homologous recombination (HR), non-homologous end-joining (NHEJ) or microhomology-mediated end-joining (MMEJ).

HR-mediated repair is a high-fidelity mechanism essential for accurate error-free repair, preventing cancer-predisposing genomic instability. Conversely, NHEJ and MMEJ are error-prone pathways that can leave mutational scars at the site of repair. MMEJ, also referred to as alternative end-joining (alt-EJ), alternative NHEJ (alt-NHEJ), and theta-mediated end-joining (TMEJ) can function in parallel to both HR and NHEJ pathways.

The survival of cancer cells, unlike normal cells, is often dependent on the mis-regulation of DNA damage response (DDR) pathways. For example, an increased dependency on one pathway (often mutagenic) to cope with either the inactivation of another one, or the enhanced replication stress resulting from increased proliferation. An aberrant DDR can also sensitise cancer cells to specific types of DNA damage, thus, defective DDR can be exploited to develop targeted cancer therapies. Crucially, cancer cells with impairment or inactivation of HR and NHEJ become hyper-dependent on MMEJ-mediated DNA repair.

With the importance of DNA repair mechanisms underscored by their critical role in healthy human physiology, their mis-regulation in a variety of disease states, and the potential for their therapeutic exploitation, it is important to have assays that measure their occurrence, integrity and efficiency in cells. From a therapeutic perspective, it is essential that these methods are titratable and sensitive to small molecule pathway inhibitors.

Approaches have been developed to measure cellular DNA repair using reporter substrates, either integrated in the genome (chromosomal), or introduced by transient transfection (extrachromosomal). They rely on the functional reconstitution of a reporter gene by a specific DNA repair mechanism and serve as a surrogate for the repair pathway. However, existing assays can require extended incubations, limited signal-to-noise ratios, poor sensitivity, restriction to specific model cell lines, limited throughput and are often labour intensive.

There is therefore a need to develop a specific, rapid, robust and quantitative assay for the detection of NHEJ and MMEJ in cells.

### SUMMARY OF THE INVENTION

According to a first aspect of the invention, there is provided a non-homologous end-joining (NHEJ) or a microhomology-mediated end joining (MMEJ) detection construct comprising:
(a) an open-reading frame (ORF) which encodes a reporter;
(b) a terminator sequence; and
(c) a promoter downstream of said terminator sequence,
wherein a portion of said reporter is downstream of said promoter and the remaining portion of said reporter is upstream of said terminator sequence.

According to a further aspect of the invention, there is provided a non-homologous end-joining (NHEJ) or a microhomology-mediated end joining (MMEJ) detection assay which comprises the following steps:
(a) providing the NHEJ or MMEJ construct as defined herein;
(b) contacting said construct with a mediator of NHEJ or MMEJ; and
(c) detecting expression of the functional reporter.

According to a further aspect of the invention, there is provided a method of screening for a modulator of NHEJ or MMEJ which comprises:
(a) providing the NHEJ or MMEJ construct as defined herein;
(b) contacting said construct with a mediator of NHEJ or MMEJ; and
(c) detecting expression of the functional reporter in both the presence and absence of a modulator of NHEJ or MMEJ,

wherein a decrease in expression relative to control is indicative of an inhibitor or antagonist of NHEJ or MMEJ and an increase in expression relative to control is indicative of an enhancer or agonist of NHEJ or MMEJ.

### BRIEF DESCRIPTION OF THE FIGURES

FIG. 1: Strategy for reporter assays. Linear DNA repair substrates comprise a non-expressible arrangement of promoter and disrupted reporter gene ORF. Upon transfection into cells, functional reconstitution of the reporter gene ORF is achieved through a specific cellular DNA repair pathway and results in functional reporter protein expression. Both intra- and inter-molecular repair are possible during reconstitution of the ORF.

FIG. 2: Schematic of the MMEJ reporter assay. The MMEJ substrate comprises a dsDNA region with flanking ssDNA overhangs containing homologous sequences at the termini. The substrate can be transiently transfected into cells, and cellular repair can occur by inter- or intra-molecular MMEJ using the terminal 4 nucleotide microhomologies on complementary DNA strands. This restores the ATG of an intact reporter gene ORF downstream of a CMV promoter, and results in the generation of a functional reporter protein. NHEJ-mediated repair of the substrate results in extensive loss of the ssDNA overhangs, including deletion of the start codon embedded in the microhomology termini, resulting in no ORF expression.

FIG. 3: Strategy for the generation of NanoLuciferase MMEJ reporter substrate. The scheme outlines the method of MMEJ substrate generation. For the NanoLuciferase-PEST version of this reporter, the dsDNA core region of the reporter is excised from a plasmid by double digestion with XhoI and HindIII. Annealed oligonucleotides form the LEFT and RIGHT caps, comprising 45 nt ssDNA overhangs with terminal 5′-ATGG/5′-CCAT microhomologies, and these are ligated to the core region via cognate XhoI and HindIII ends. Enzyme choice and cap annealing oligonucleotides are determined by the sequence of the ORF (e.g. NanoLuciferase).

FIG. 4: Generation of the NanoLuciferase MMEJ reporter DNA Substrate. (a) Pairs of oligonucleotides (length shown in superscript) were annealed to form LEFT (L) and RIGHT (R) annealed caps (LA, RA) with terminal stretches of 45 nt ssDNA encoding terminal microhomology. (b) The caps were ligated to the XhoI/Hind/III-digested core dsDNA fragment with or without Antarctic phosphatase (AP) treatment. AP treatment ensures complete ligation to generate the substrate. Excess cap DNA was removed by gel purification. Enzyme choice is determined by the sequence of the ORF (e.g. NanoLuciferase).

FIG. 5: NanoLuciferase activity is observed after MMEJ-mediated repair only. HEK293 cells were transfected with linear DNA substrates mimicking the products of NHEJ- or MMEJ-mediated repair. Data represent mean+SEM of n=3 (technical replicates), analysed by one-way ANOVA with Tukey's multiple comparisons; **** =p<0.0001, ns=not significant. NanoLuciferase luminescence was strictly dependent on the intact ORF (MMEJ product).

FIG. 6: NanoLuciferase activity following NanoLuc MMEJ reporter DNA substrate transfection is inhibited by DNA polymerase 8 inhibitors in a concentration-dependent manner. HEK293 cells transfected with the NanoLuc MMEJ Reporter DNA Substrate and a Firefly luciferase plasmid (transfection control) were treated with increasing concentrations of Compound A (a) or Compound B (b). NanoLuciferase luminescence was normalised to FireFly luminescence, and percentage inhibition was calculated relative to the DMSO control. Data represent mean±SEM of n=3 (technical replicates), fitted with a non-linear regression curve (four parameters). Data and EC50s from two independent experiments are shown.

FIG. 7: MMEJ-mediated repair of the NanoLuc MMEJ reporter DNA substrate is reduced upon DNA polymerase 8 inhibition. Cells transfected with the NanoLuc MMEJ. Reporter DNA substrate were treated with DMSO or 6 μM Compound C. (a) gDNA was harvested, and products of MMEJ and NHEJ were amplified by PCR (MMEJ and NHEJ control plasmids were included as size markers for MMEJ and NHEJ products). PCR products were visualised on a polyacrylamide gel. (b) Band intensity measurements enabled quantification of the MMEJ product relative to the NHEJ product.

FIG. 8: Schematic of the NHEJ reporter DNA assay. The blunt NHEJ substrate can be excised by restriction digest with a blunt-cutting restriction enzyme, “RE”, (e.g. EcoRV) to generate a linear DNA molecular with blunt ends that can be transiently transfected into cells. Cellular repair by direct ligation NHEJ restores the reporter gene ORF and the generation of functional reporter protein.

FIG. 9: Generation of the NanoLuciferase NHEJ reporter DNA substrate. The blunt NHEJ substrate can be excised by restriction digest with a blunt-cutting restriction enzyme, EcoRV, to generate a linear DNA molecular with blunt ends. After separation by gel electrophoresis and purification by gel extraction, the substrate can be transiently transfected into cells. Cellular repair by direct ligation NHEJ restores the reporter gene ORF and the generation of functional reporter protein.

FIG. 10: NHEJ-mediated repair of the NanoLuciferase NHEJ reporter DNA substrate is reduced in NHEJ-deficient genetic backgrounds and upon compound-mediated inhibition of NHEJ.

(A) Wild-type or NHEJ-deficient XRCC4^{-/-}, XLF^{-/-}) cells were transfected with the NanoLuc NHEJ Reporter DNA substrate and a Firefly luciferase plasmid (transfection control). NHEJ repair efficiency was expressed as NanoLuciferase luminescence normalised to FireFly luminescence (arbitrary units). Data represent mean+SEM of n=4 (technical replicates), analysed by one-way ANOVA with Dunnett's multiple comparisons; **** =p<0.0001. (B) Cells were pre-incubated with indicated concentrations of NU7441, an inhibitor of NHEJ machinery component DNA-PKcs, and then transfected with the NHEJ reporter. NanoLuciferase luminescence was normalised to FireFly luminescence, and percentage inhibition was calculated relative to the DMSO control. Data represent mean±SEM of n=3 (technical replicates).

FIG. 11: NanoLuciferase activity following NanoLuc MMEJ reporter DNA substrate transfection is inhibited by an active, but not an inactive, DNA polymerase 8 inhibitor. HEK293 cells transfected with the NanoLuc MMEJ Reporter DNA Substrate and a Firefly luciferase plasmid (transfection control) were treated with increasing concentrations of active Compound A (closed circles, line) or inactive Compound D (open squares, dashed line). NanoLuciferase luminescence was normalised to FireFly luminescence, and percentage inhibition was calculated relative to the DMSO control. Data represent mean±SD of n=4 (technical replicates), fitted with a non-linear regression curve (four parameters). Data and EC50s from two independent experiments are shown (Experiment 1, upper panel and Experiment 2, lower panel).

### DETAILED DESCRIPTION OF THE INVENTION

Constructs

According to a first aspect of the invention, there is provided a non-homologous end-joining (NHEJ) or a microhomology-mediated end joining (MMEJ) detection construct comprising:
(a) an open-reading frame (ORF) which encodes a reporter;
(b) a terminator sequence; and
(c) a promoter downstream of said terminator sequence, wherein a portion of said reporter is downstream of said promoter and the remaining portion of said reporter is upstream of said terminator sequence.

The inventors have surprisingly generated two novel DNA substrates that can be transfected into cells to detect either NHEJ or MMEJ and thus serve as a cellular reporter for the efficiency of these repair mechanisms.

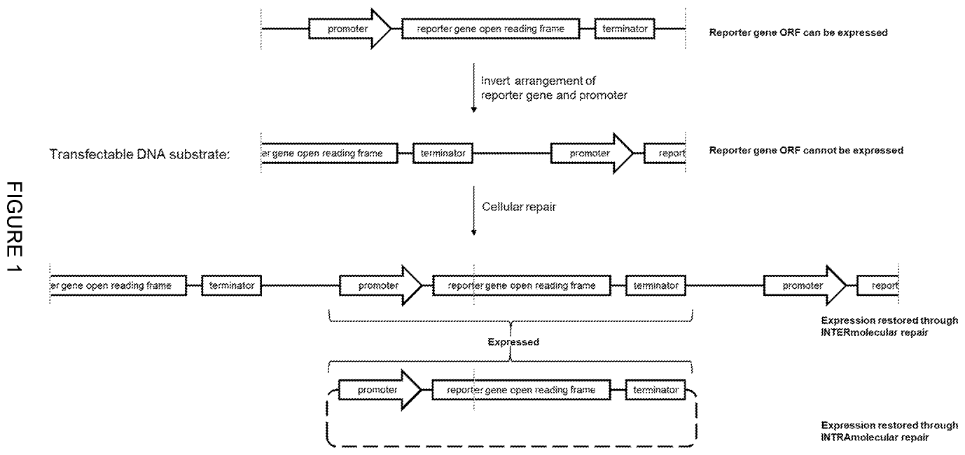

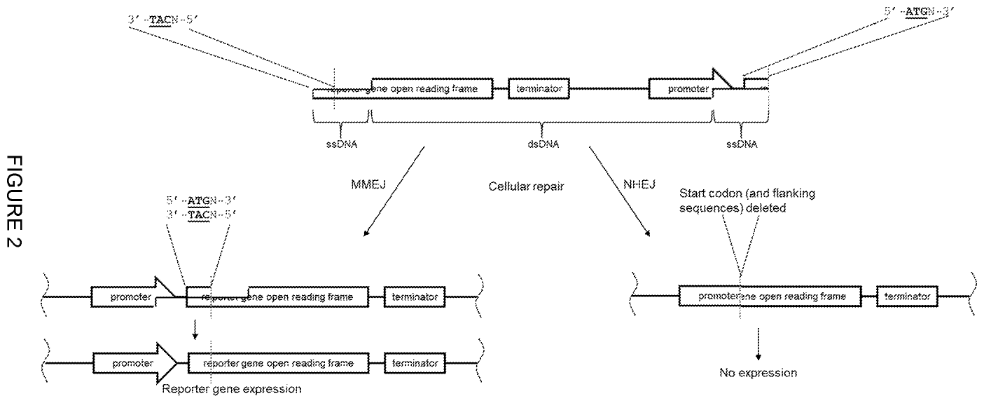

The nucleic acid construct is a linear DNA substrate comprising a non-expressible arrangement of a promoter and disrupted reporter gene open reading frame, as indicated in FIG. 1. Intra- or inter-molecular functional reconstitution of the reporter gene ORF is achieved by a specific cellular DNA repair pathway (e.g. NHEJ or MMEJ) placing the intact complete ORF downstream of the promoter, permitting generation of the functional reporter protein.

The resultant assay comprising these constructs has been found to be surprisingly sensitive to small molecule pathway inhibitors, titratable, and can be adapted to a high throughput format amenable to inhibitor screening.

References herein to the term “NHEJ detection construct” refer to a nucleic acid molecule (typically DNA) which comprises components which allow said nucleic acid molecule to provide a read-out (via detection means) regarding whether NHEJ has been successfully conducted (i.e. classifying the integrity of DNA repair in cells to be either proficient or deficient).

References herein to the term “MMEJ detection construct” refer to a nucleic acid molecule (typically DNA) which comprises components which allow said nucleic acid molecule to provide a read-out (via detection means) regarding whether MMEJ has been successfully conducted (i.e. classifying the integrity of DNA repair in cells to be either proficient or deficient).

References herein to the term “open reading frame” refer to the part of a reading frame that has the ability to be translated. An ORF is generally a continuous stretch of codons that begins with a start codon (typically AUG) and ends at a stop codon (typically UAA, UAG or UGA).

References herein to the term “reporter” refer to any entity (i.e. protein) suitable for being easily identified and measured and is typically a detectable (i.e. reporter) protein. The ORF can be used to encode any relevant protein, which can be inert (e.g. fluorescent or luminescent) or functional (e.g. survival) in nature. In the former case, these reporters can be generated for high throughput screening formats using luciferase readouts (detectable by a plate reader) or fluorophore readouts (detectable by imaging or flow cytometry methods) with or without destabilisation domains. In the latter case, these reporters can be generated for genetic survival screens, for example through the use of a negative selection marker such as HSV-TK.

The use of fluorescent or luminescent proteins in cellular reporters is not new, and the most widely used chromosomal reporters of HR, NHEJ and MMEJ use functional reconstitution of GFP by the aforementioned DNA repair pathways to generate a fluorescent signal detectable by flow cytometry. The advantage of luciferase derivatives over fluorophores is that they are detected through catalytic evolution of luminescent fumaramide from the substrate furimazine, allowing signal amplification and detection from cells in situ through a plate reader equipped for luminescence detection.

Suitable non-limiting examples of reporters include: β-galactosidase (IacZ), chloramphenicol acetyltransferase (cat) and fluorescent or luminescent proteins, such as green fluorescent protein (gfp), red fluorescent protein (rfp) or NanoLuciferase. In one embodiment, the reporter is selected from a fluorescent or a luminescent protein, such as GFP or NanoLuciferase.

In a further embodiment, the reporter is NanoLuciferase. NanoLuciferase is a recently described engineered luciferase enzyme. This 19 kDa protein is brighter than Firefly or Renilla luciferase, has excellent thermal and pH tolerability for in vitro cellular studies, and has low autoluminescence thereby enhancing assay sensitivity. The low molecular weight reduces the size of the transfected DNA substrate, and the brightness permits signal detection from cell assays scaled down to 96- and 384-well plates.

These properties ensure that quantifiable detection of NanoLuciferase is possible even at very low expression levels, which is critical for the detection of infrequent repair events (such as NHEJ and MMEJ) under endogenous control in cells. The inclusion of a protein destabilisation domain (PEST sequence) on the reporter sequence can also be considered to detect faster and more sensitive responses to stimulatory activation and reduce effects of cellular accumulation of NanoLuciferase.

The use of direct detection of cellular NanoLuciferase expression in situ improves upon the processing methods of cells that can limit throughput for flow cytometry-based detection of fluorescent reporters.

References herein to the term “terminator sequence” refer to a section of a nucleic acid sequence that marks the end of a gene or operon of genomic DNA during transcription. In one embodiment, the terminator sequence is selected from a mammalian terminator sequence and includes a polyadenylation signal, such as SV40, hGH, BGH and rbGlob.

References herein to the term “promoter” refer to a section of a nucleic acid sequence that initiates transcription of a particular gene. In one embodiment, the promoter is selected from a cytomegalovirus (CMV) or an SV40 promoter. In a further embodiment, the promoter is a CMV promoter.

References herein to the terms “upstream” and “downstream” refer to a given element which is closer to the 5′ to 3′ direction, respectively, in which RNA transcription takes place relative to a reference element. Typically, upstream is toward the 5′ end of the RNA molecule and downstream is toward the 3′ end. When considering double-stranded DNA, upstream is toward the 5′ end of the coding strand for the gene in question and downstream is toward the 3′ end.

It will be appreciated that any portion of said reporter may be present downstream of said promoter. The key aspect of the invention is that the reporter is “split” and contains a portion downstream of the promoter and the remaining portion upstream of said terminator sequence. Generally, most of said reporter will be present upstream of said terminator and only a small portion of the promoter will be present downstream of the promoter. In one embodiment, the portion of said reporter which is downstream of said promoter comprises the first 2 to 10 nucleotides which encode said reporter, such as the first 2 to 8 nucleotides, in particular the first 2 to 7 nucleotides, especially the first 4 nucleotides (i.e. ATGX), more especially the first 3 nucleotides, e.g. the start codon of said reporter (i.e. ATG).

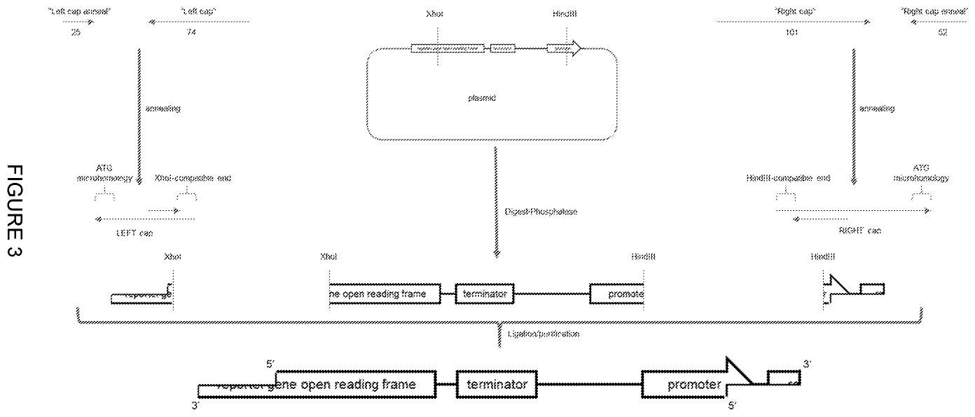

It will be appreciated from the schematic drawings of FIGS. 2 and 8 that the key distinction between the NHEJ and MMEJ detection constructs relates to whether the portion of said reporter which is downstream of said promoter is blunt ended (NHEJ) or single stranded and containing microhomologies (MMEJ).

NHEJ Constructs

Thus, in one embodiment, the detection construct is a NHEJ detection construct and the portion of said reporter which is downstream of said promoter and the remaining portion of said reporter which is upstream of said terminator sequence are both blunt ended.

According to a further aspect of the invention, there is provided an NHEJ detection construct comprising:
(a) an open-reading frame (ORF) which encodes a reporter;
(b) a terminator sequence; and
(c) a promoter downstream of said terminator sequence,

wherein a portion of said reporter is downstream of said promoter and the remaining portion of said reporter is upstream of said terminator sequence, characterised in that the portion of said reporter which is downstream of said promoter and the remaining portion of said reporter which is upstream of said terminator sequence are both blunt ended.

NHEJ involves the joining of both ends of a DSB using partial processing and ligation. Many proteins have been described to be involved at each stage of the process, including end recognition (e.g. Ku70/Ku80 and DNA-PKcs), end-processing (which may not be invoked if ends are compatible and/or correctly terminated) and ligation (XLF, XRCC4 and Ligase IV). NHEJ is largely restricted to DSB repair events when no homologous template is available for the error-free process of HR.

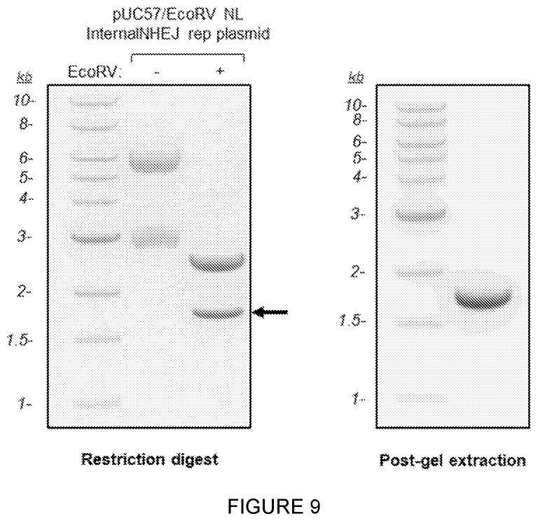

The layout of the reporter is outlined in FIG. 8. The substrate has been engineered to express a functional reporter only when re-ligation of unprocessed blunt ends has been correctly performed. A blunt-cutting restriction enzyme site can be silently engineered in the ORF, and the locus can then be rearranged as indicated and synthesised. In vitro excision of the reporter from a backbone vector by the blunt-cutting restriction enzyme generates the linear transfectable reporter (FIG. 9). Upon transfection, these blunt ends must be brought together and re-ligated by components of the cellular NHEJ machinery, without degradation of the encoded ORF sequence, to restore a functional ORF downstream of the promoter. The unrepaired reporter cannot express functional protein as the encoded gene has been split and only the N-terminus is expressed under the promoter.

MMEJ Constructs

In an alternative embodiment, the detection construct is an MMEJ detection construct and the portion of said reporter which is downstream of said promoter is single stranded.

Thus, according to a further aspect of the invention, there is provided an MMEJ detection construct comprising:
(a) an open-reading frame (ORF) which encodes a reporter;
(b) a terminator sequence; and
(c) a promoter downstream of said terminator sequence,

wherein a portion of said reporter is downstream of said promoter and the remaining portion of said reporter is upstream of said terminator sequence, characterised in that the portion of said reporter which is downstream of said promoter is single stranded.

For example, said single stranded portion of said reporter which is downstream of said promoter comprises the first 2 to 10 nucleotides which encode said reporter, such as the first 2 to 8 nucleotides, in particular the first 2 to 7 nucleotides, especially the first 4 nucleotides (i.e. ATGX), more especially the first 3 nucleotides, e.g. the start codon of said reporter (i.e. ATG). In a further embodiment, the terminal portion of the promoter is also single stranded.

In one embodiment, the detection construct is an MMEJ detection construct and the portion of said reporter which is upstream of said terminator sequence is double stranded with a 3′ single stranded overhang. In a further embodiment, the 3′ single stranded overhang comprises the reverse compliment of the first 100 nucleotides of said reporter, such as the first 50 nucleotides, in particular the first 45 nucleotides.

MMEJ involves the resection of both ends of a DSB to expose 3′ overhangs with short microhomologies that can pair by complementary annealing and align the break. The establishment of this annealed microhomology serves as a platform for 5′-3′ DNA polymerisation to fill in the remaining gaps.

Genetic, cell biological and biochemical data have identified Polθ as the key protein in MMEJ. Polθ is a multifunctional enzyme, which comprises an N-terminal helicase domain (SF2 HEL308-type) and a C-terminal low-fidelity DNA polymerase domain (A-type). Both domains have been shown to have concerted mechanistic functions in MMEJ. The helicase domain mediates the removal of RPA protein from ssDNA ends and stimulates annealing. The polymerase domain extends the ssDNA ends and fills the remaining gaps.

It has previously been reported that the efficiency of MMEJ can be determined using an extrachromosomal repair substrate (Wyatt et al (2016) Molecular Cell 63(4), 662-673). This substrate comprises a region of dsDNA flanked by two 3′ ssDNA overhangs each ending in 4 nucleotides of complementary microhomology. Transfection of the substrate into cells is followed by a 1-2 hour incubation to allow MMEJ repair to take place. Cellular gDNA can then be isolated, and a polymerase chain reaction (PCR) across the break can be used to establish whether repair proceeded through MMEJ at the overhang termini, or by nucleolytic cleavage of the overhangs and classical NHEJ bringing the two dsDNA junctions together. MMEJ repair produces a longer product as the repaired substrate includes the polymerase filled-in overhangs. NHEJ produces a shorter product as the overhangs have been removed.

The use of this extrachromosomal MMEJ substrate has been used to characterise the genetic requirements of MMEJ in isogenic backgrounds (e.g. Polθ and Ku70 knockouts) but it is labour intensive, and has not been demonstrated to be titratable, robustly quantitative, or amenable to a high throughout format as it requires a PCR and gel electrophoresis readout.

To circumvent these issues, the inventors have developed a novel DNA substrate that functions as a cellular MMEJ reporter. This substrate specifically relies on MMEJ-mediated repair to reconstitute the open reading frame of a reporter gene.

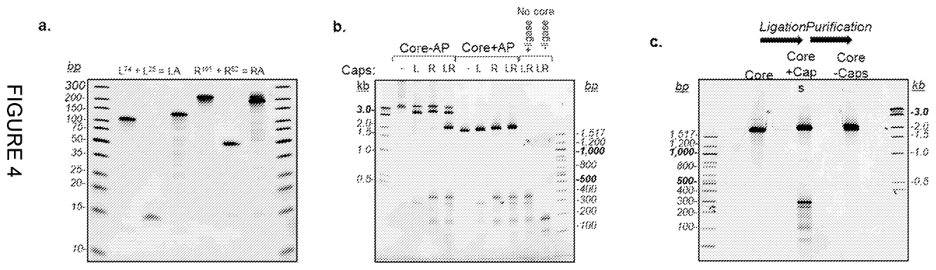

The layout of the reporter is outlined in FIG. 2. The substrate has been engineered to express a functional reporter protein only when MMEJ has been correctly performed. For expression of a gene, it must be downstream of a functional promoter. By inverting this arrangement, the gene is upstream of the promoter and is not expressed. In order to make this an MMEJ reporter, restriction sites have been engineered into the nucleic acid sequence, comprising ORF-terminator-promoter-linker. Annealed oligonucleotide adapters are then ligated on to the ends, via complementary ends, yielding 45-nt ssDNA overhangs with 4 nt microhomology at the termini (FIG. 3).

The use of MMEJ repair, via the terminal 4-nt of microhomology, places the ORF downstream of the promoter and restores the initiator ATG codon (embedded in the microhomology) necessary for expression of the full-length reporter protein.

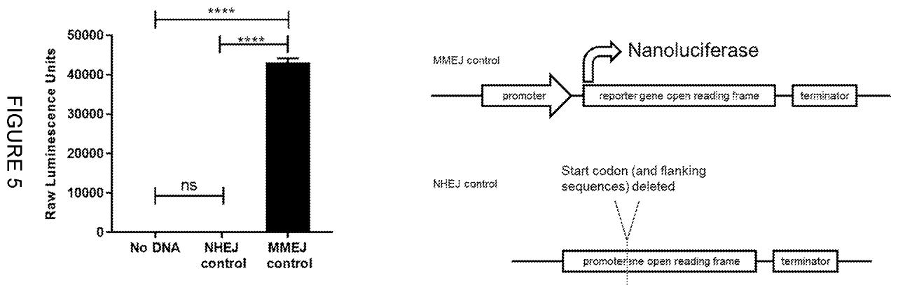

The example data utilises the NanoLuciferase-PEST protein as the reporter encoded by the ORF. The strategy for substrate generation is outlined in FIG. 3. The locus was gene synthesised to provide the arrangement shown, placing the NanoLuciferase gene upstream of a CMV promoter. Silent mutations introduced a XhoI restriction site in the NanoLuciferase gene and ensured a single HindIII site in the promoter. Oligonucleotides were annealed (FIG. 4A) to generate ssDNA/dsDNA caps with a 45 nt ssDNA overhang containing 4 nt terminal microhomology (5′-ATGG (right)/5′-CCAT (left)) and a XhoI (left) or HindIII (right) complementary overhang.

The dsDNA substrate fragment excised by XhoI and HindIII (“core”) from a parental vector, was phosphatase-treated and ligated to the right and left caps (FIGS. 4B and 4C) to generate a single MMEJ substrate species. Excess, unligated cap DNA was separated from the substrate by gel purification (FIG. 4C).

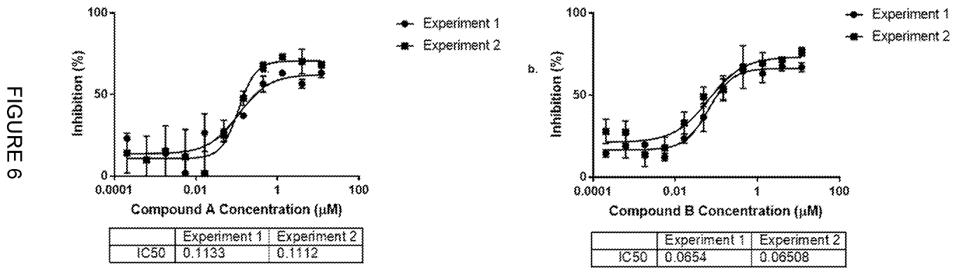

The linear substrate can be electroporated into cells using standard conditions. To check for specificity of the reporter signal, a DNA fragment expressing a repair product mimicking terminal MMEJ repair or NHEJ repair was electroporated into HEK293 cells (FIG. 5). Only the MMEJ product produced a detectable NanoLuciferase signal, suggesting that repair by MMEJ was necessary for functional reporter expression by placing the intact ORF downstream of the promoter.

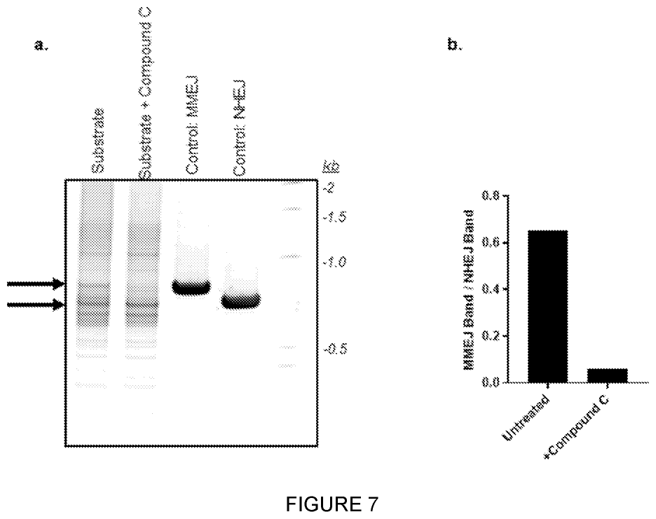

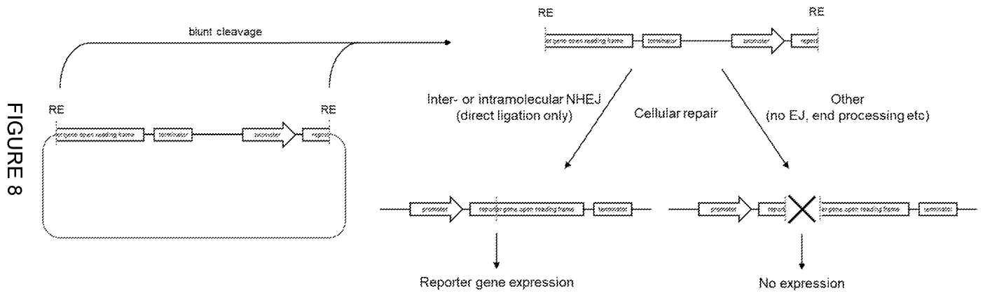

To demonstrate compound sensitivity of the MMEJ reporter, HEK293 cells were electroporated with the MMEJ substrate and subsequently plated to validated Polθ inhibitor (Polθi) Compounds A and B, prediluted in a 12-point 3-fold dilution series. In two independent experiments, MMEJ repair was detectable, compound-sensitive and titratable (FIG. 6), generating consistent EC50s. To ensure that the signal detected was because of a direct effect on DNA, genomic DNA was prepared from cells treated with DMSO or Polθi Compound C. A PCR across the termini revealed that the MMEJ product was significantly reduced following compound treatment, supporting a direct and specific effect of the compound on MMEJ-mediated DNA repair (FIG. 7).

To demonstrate the specificity of the compound sensitivity of the MMEJ reporter, HEK293 cells were electroporated with the MMEJ substrate and subsequently plated to validated Polθ inhibitor (Polθi) Compound A or an inactive Compound D, prediluted in a 9-point 3-fold dilution series. In two independent experiments, MMEJ repair was specifically inhibited by the active compound, but not the inactive compound (FIG. 11) demonstrating that the reporter substrate can be used to discriminate the highly specific, selective and titratable pharmacological inhibition of a key mediator of MMEJ (Polθi) whilst remaining unaffected by a structurally-related but inactive compound.

Detection Assays

According to a further aspect of the invention, there is provided a non-homologous end-joining (NHEJ) or a microhomology-mediated end joining (MMEJ) detection assay which comprises the following steps:
(a) providing the NHEJ or MMEJ construct as defined herein;
(b) contacting said construct with a mediator of NHEJ or MMEJ; and
(c) detecting expression of the functional reporter.

According to a further aspect of the invention, there is provided an NHEJ detection assay which comprises the following steps:
(a) providing the NHEJ construct as defined herein;
(b) contacting said construct with a mediator of NHEJ; and
(c) detecting expression of the functional reporter.

According to a further aspect of the invention, there is provided an MMEJ detection assay which comprises the following steps:
(a) providing the MMEJ construct as defined herein;
(b) contacting said construct with a mediator of MMEJ; and
(c) detecting expression of the functional reporter.

References herein to “mediator of NHEJ” or “mediator of MMEJ” refer to any component or components within the NHEJ or MMEJ machinery.

In one embodiment, the mediator of MMEJ comprises Polθ. Full details of Polθ and its function are described hereinbefore.

Screening Methods

According to a further aspect of the invention, there is provided a method of screening for a modulator of NHEJ or MMEJ which comprises:
(a) providing the NHEJ or MMEJ construct as defined herein;

(b) contacting said construct with a mediator of NHEJ or MMEJ; and

(c) detecting expression of the functional reporter in both the presence and absence of a modulator of NHEJ or MMEJ,

wherein a decrease in expression relative to control is indicative of an inhibitor or antagonist of NHEJ or MMEJ and an increase in expression relative to control is indicative of an enhancer or agonist of NHEJ or MMEJ.

According to a further aspect of the invention, there is provided a method of screening for a modulator of NHEJ which comprises:
(a) providing the NHEJ construct as defined herein;
(b) contacting said construct with a mediator of NHEJ; and
(c) detecting expression of the functional reporter in both the presence and absence of a modulator of NHEJ,

wherein a decrease in expression relative to control is indicative of an inhibitor or antagonist of NHEJ and an increase in expression relative to control is indicative of an enhancer or agonist of NHEJ.

References herein to “modulator of NHEJ” refer to any entity (i.e. small molecule, nucleic acid, protein and the like) which is capable of effecting modulation (i.e. inhibition, antagonism, enhancement or agonism) of NHEJ.

According to a further aspect of the invention, there is provided a method of screening for a modulator of MMEJ which comprises:
(a) providing the MMEJ construct as defined herein;
(b) contacting said construct with a mediator of MMEJ; and
(c) detecting expression of the functional reporter in both the presence and absence of a modulator of MMEJ,

wherein a decrease in expression relative to control is indicative of an inhibitor or antagonist of MMEJ and an increase in expression relative to control is indicative of an enhancer or agonist of MMEJ.

References herein to “modulator of MMEJ” refer to any entity (i.e. small molecule, nucleic acid, protein and the like) which is capable of effecting modulation (i.e. inhibition, antagonism, enhancement or agonism) of MMEJ.

In one embodiment, the modulator of MMEJ comprises a Polθ inhibitor.

According to a further aspect of the invention, there is provided a Polθ inhibitor identified in accordance with a screening method described herein.

The following non-limiting studies illustrate the invention:

Materials And Methods

Substrate Generation

The generation of the example MMEJ substrate is outlined in FIGS. 3 and 4. The vector denoted as “pMK-RQ-NLcoreMMEJreporter”, comprising a CMV promoter downstream of the NanoLuciferase-PEST gene (hereafter referred to as NanoLuciferase) and SV40 poly A terminator, was generated by gene synthesis (GeneArt) (Table 1). The sequences were derived, and rearranged, from pNL3.2CMV (Promega) with the addition of the incorporation of silent nucleotide substitutions introducing a XhoI restriction site into the NanoLuciferase coding region and eliminating a second HindIII site from the multiple cloning site.

A region of the vector was excised by restriction digest with XhoI and HindIII enzymes (Table 1), followed by dephosphorylation with Antarctic Phosphatase (NEB). The core substrate fragment was separated from the vector backbone by agarose gel electrophoresis and purified by gel extraction (Qiagen).

TABLE 1
Plasmid sequences

| Plasmid | Sequence |
|---|---|
| Complete | CTAAATTGTAAGCGTTAATATTTTGTTAAAATTCGCGTTAAATTTTTGTT |
| plasmid | AAATCAGCTCATTTTTTAACCAATAGGCCG |
| sequence of | AAATCGGCAAAATCCCTTATAAATCAAAAGAATAGACCGAGATAGGGT |
| pMK-RQ- | TGAGTGGCCGCTACAGGGCGCTCCCATTCGCC |
| NLcoreMME | ATTCAGGCTGCGCAACTGTTGGGAAGGGCGTTTCGGTGCGGGCCTCT |
| Jreporter. | TCGCTATTACGCCAGCTGGCGAAAGGGGGATGT |
| The core | GCTGCAAGGCGATTAAGTTGGGTAACGCCAGGGTTTTCCCAGTCACG |
| substrate | ACGTTGTAAAACGACGGCCAGTGAGCGCGACGT |
| fragment | AATACGACTCACTATAGGGCGAATTGAAGGAAGGCCGTCAAGGCCGC |
| excised by | ATATGGTCTTCACACTCGAAGATTTCGTTGGGG |
| Xhol and | ACTGGCGACAGACAGCCGGCTACAACCTGGACCAAGTCCTCGAGCA |
| HindIII | GGGAGGTGTGTCCAGTTTGTTTCAGAATCTCGGG |
| digest is in | GTGTCCGTAACTCCGATCCAAAGGATTGTCCTGAGCGGTGAAAATGG |
| italics. | GCTGAAGATCGACATCCATGTCATCATCCCGTA |
| Enzyme | TGAAGGTCTGAGCGGCGACCAAATGGGCCAGATCGAAAAAATTTTTA |
| recognition | AGGTGGTGTACCCTGTGGATGATCATCACTTTA |
| sites are | AGGTGATCCTGCACTATGGCACACTGGTAATCGACGGGGTTACGCCG |
| underlined. | AACATGATCGACTATTTCGGACGGCCGTATGAA |
| | GGCATCGCCGTGTTCGACGGCAAAAAGATCACTGTAACAGGGACCCT |
| | GTGGAACGGCAACAAAATTATCGACGAGCGCCT |
| | GATCAACCCCGACGGCTCCCTGCTGTTCCGAGTAACCATCAACGGAG |
| | TGACCGGCTGGCGGCTGTGCGAACGCATTCTGG |
| | CGAATTCTCACGGCTTTCCGCCTGAGGTTGAAGAGCAAGCCGCCGGT |
| | ACATTGCCTATGTCCTGCGCACAAGAAAGCGGT |
| | ATGGACCGGCACCCAGCCGCTTGTGCTTCAGCTCGCATCAACGTCTA |
| | AGGCCGCGACTCTAGAGTCGGGGCGGCCGGCCG |
| | CTTCGAGCAGACATGATAAGATACATTGATGAGTTTGGACAAACCACA |
| | ACTAGAATGCAGTGAAAAAAATGCTTTATTTG |
| | TGAAATTTGTGATGCTATTGCTTTATTTGTAACCATTATAAGCTGCAAT |
| | AAACAAGTTAACAACAACAATTGCATTCATT |
| | TTATGTTTCAGGTTCAGGGGGAGGTGTGGGAGGTTTTTTAAAGCAAGT |
| | AAAACCTCTACAAATGTGGTAAAATCGATAAG |
| | GATCCGTTTGCGTATTGGGCGCTCTTCCGCTGATCTGGCCTAACTGG |
| | CCTCAATATTGGCCATTAGCCATATTATTCATT |
| | GGTTATATAGCATAAATCAATATTGGCTATTGGCCATTGCATACGTTGT |
| | ATCTATATCATAATATGTACATTTATATTGG |
| | CTCATGTCCAATATGACCGCCATGTTGGCATTGATTATTGACTAGTTAT |
| | TAATAGTAATCAATTACGGGGTCATTAGTTC |
| | ATAGCCCATATATGGAGTTCCGCGTTACATAACTTACGGTAAATGGCC |
| | CGCCTGGCTGACCGCCCAACGACCCCCGCCCA |
| | TTGACGTCAATAATGACGTATGTTCCCATAGTAACGCCAATAGGGACT |
| | TTCCATTGACGTCAATGGGTGGAGTATTTACG |
| | GTAAACTGCCCACTTGGCAGTACATCAAGTGTATCATATGCCAAGTCC |
| | GCCCCCTATTGACGTCAATGACGGTAAATGGC |
| | CCGCCTGGCATTATGCCCAGTACATGACCTTACGGGACTTTCCTACTT |
| | GGCAGTACATCTACGTATTAGTCATCGCTATT |
| | ACCATGGTGATGCGGTTTTGGCAGTACACCAATGGGCGTGGATAGCG |
| | GTTTGACTCACGGGGATTTCCAAGTCTCCACCC |
| | CATTGACGTCAATGGGAGTTTGTTTTGGCACCAAAATCAACGGGACTT |
| | TCCAAAATGTCGTAATAACCCCGCCCCGTTGA |
| | CGCAAATGGGCGGTAGGCGTGTACGGTGGGAGGTCTATATAAGCAGA |
| | GCTCGTTTAGTGAACCGTCAGATCACTAGAAGC |
| | TTTATTGCGGTAGTTTATCACAGTTAAATTGCTAACGCAGTCAGTGGG |
| | CCTCGGCGGCCAAGCTAGGCAATCCGGTACTG |
| | TTGGTAAAGCCACCATGGTGAGCTAACGTAGCTGGGCCTCATGGGCC |
| | TTCCTTTCACTGCCCGCTTTCCAGTCGGGAAAC |
| | CTGTCGTGCCAGCTGCATTAACATGGTCATAGCTGTTTCCTTGCGTAT |
| | TGGGCGCTCTCCGCTTCCTCGCTCACTGACTC |
| | GCTGCGCTCGGTCGTTCGGGTAAAGCCTGGGGTGCCTAATGAGCAAA |
| | AGGCCAGCAAAAGGCCAGGAACCGTAAAAAGGC |
| | CGCGTTGCTGGCGTTTTTCCATAGGCTCCGCCCCCCTGACGAGCATC |
| | ACAAAAATCGACGCTCAAGTCAGAGGTGGCGAA |
| | ACCCGACAGGACTATAAAGATACCAGGCGTTTCCCCCTGGAAGCTCC |
| | CTCGTGCGCTCTCCTGTTCCGACCCTGCCGCTT |
| | ACCGGATACCTGTCCGCCTTTCTCCCTTCGGGAAGCGTGGCGCTTTC |
| | TCATAGCTCACGCTGTAGGTATCTCAGTTCGGT |
| | GTAGGTCGTTCGCTCCAAGCTGGGCTGTGTGCACGAACCCCCCGTTC |
| | AGCCCGACCGCTGCGCCTTATCCGGTAACTATC |
| | GTCTTGAGTCCAACCCGGTAAGACACGACTTATCGCCACTGGCAGCA |
| | GCCACTGGTAACAGGATTAGCAGAGCGAGGTAT |
| | GTAGGCGGTGCTACAGAGTTCTTGAAGTGGTGGCCTAACTACGGCTA |
| | CACTAGAAGAACAGTATTTGGTATCTGCGCTCT |
| | GCTGAAGCCAGTTACCTTCGGAAAAAGAGTTGGTAGCTCTTGATCCG |
| | GCAAACAAACCACCGCTGGTAGCGGTGGTTTTT |
| | TTGTTTGCAAGCAGCAGATTACGCGCAGAAAAAAAGGATCTCAAGAAG |
| | ATCCTTTGATCTTTTCTACGGGGTCTGACGCT |
| | CAGTGGAACGAAAACTCACGTTAAGGGATTTTGGTCATGAGATTATCA |
| | AAAAGGATCTTCACCTAGATCCTTTTAAATTA |
| | AAAATGAAGTTTTAAATCAATCTAAAGTATATATGAGTAAACTTGGTCT |
| | GACAGTTATTAGAAAAATTCATCCAGCAGAC |
| | GATAAAACGCAATACGCTGGCTATCCGGTGCCGCAATGCCATACAGC |
| | ACCAGAAAACGATCCGCCCATTCGCCGCCCAGT |
| | TCTTCCGCAATATCACGGGTGGCCAGCGCAATATCCTGATAACGATCC |
| | GCCACGCCCAGACGGCCGCAATCAATAAAGCC |
| | GCTAAAACGGCCATTTTCCACCATAATGTTCGGCAGGCACGCATCACC |
| | ATGGGTCACCACCAGATCTTCGCCATCCGGCA |
| | TGCTCGCTTTCAGACGCGCAAACAGCTCTGCCGGTGCCAGGCCCTGA |
| | TGTTCTTCATCCAGATCATCCTGATCCACCAGG |
| | CCCGCTTCCATACGGGTACGCGCACGTTCAATACGATGTTTCGCCTG |
| | ATGATCAAACGGACAGGTCGCCGGGTCCAGGGT |
| | ATGCAGACGACGCATGGCATCCGCCATAATGCTCACTTTTTCTGCCG |
| | GCGCCAGATGGCTAGACAGCAGATCCTGACCCG |
| | GCACTTCGCCCAGCAGCAGCCAATCACGGCCCGCTTCGGTCACCACA |
| | TCCAGCACCGCCGCACACGGAACACCGGTGGTG |
| | GCCAGCCAGCTCAGACGCGCCGCTTCATCCTGCAGCTCGTTCAGCGC |
| | ACCGCTCAGATCGGTTTTCACAAACAGCACCGG |
| | ACGACCCTGCGCGCTCAGACGAAACACCGCCGCATCAGAGCAGCCA |
| | ATGGTCTGCTGCGCCCAATCATAGCCAAACAGAC |
| | GTTCCACCCACGCTGCCGGGCTACCCGCATGCAGGCCATCCTGTTCA |
| | ATCATACTCTTCCTTTTTCAATATTATTGAAGC |
| | ATTTATCAGGGTTATTGTCTCATGAGCGGATACATATTTGAATGTATTT |
| | AGAAAAATAAACAAATAGGGGTTCCGCGCAC |
| | ATTTCCCCGAAAAGTGCCAC (SEQ ID NO: 1) |
| Complete | CTAAATTGTAAGCGTTAATATTTTGTTAAAATTCGCGTTAAATTTTTGTT |
| plasmid | AAATCAGCTCATTTTTTAACCAATAGGCCG |
| sequence of | AAATCGGCAAAATCCCTTATAAATCAAAAGAATAGACCGAGATAGGGT |
| pMK- | TGAGTGGCCGCTACAGGGCGCTCCCATTCGCC |
| RQ/N L-pos- | ATTCAGGCTGCGCAACTGTTGGGAAGGGCGTTTCGGTGCGGGCCTCT |
| ctrl-MMEJ | TCGCTATTACGCCAGCTGGCGAAAGGGGGATGT |
| | GCTGCAAGGCGATTAAGTTGGGTAACGCCAGGGTTTTCCCAGTCACG |
| | ACGTTGTAAAACGACGGCCAGTGAGCGCGACGT |
| | AATACGACTCACTATAGGGCGAATTGAAGGAAGGCCGTCAAGGCCGC |
| | ATGTTTAAACGCGGCCGCGGCCTAACTGGCCTC |
| | AATATTGGCCATTAGCCATATTATTCATTGGTTATATAGCATAAATCAAT |
| | ATTGGCTATTGGCCATTGCATACGTTGTAT |
| | CTATATCATAATATGTACATTTATATTGGCTCATGTCCAATATGACCGC |
| | CATGTTGGCATTGATTATTGACTAGTTATTA |
| | ATAGTAATCAATTACGGGGTCATTAGTTCATAGCCCATATATGGAGTTC |
| | CGCGTTACATAACTTACGGTAAATGGCCCGC |
| | CTGGCTGACCGCCCAACGACCCCCGCCCATTGACGTCAATAATGACG |
| | TATGTTCCCATAGTAACGCCAATAGGGACTTTC |
| | CATTGACGTCAATGGGTGGAGTATTTACGGTAAACTGCCCACTTGGCA |
| | GTACATCAAGTGTATCATATGCCAAGTCCGCC |
| | CCCTATTGACGTCAATGACGGTAAATGGCCCGCCTGGCATTATGCCC |
| | AGTACATGACCTTACGGGACTTTCCTACTTGGC |
| | AGTACATCTACGTATTAGTCATCGCTATTACCATGGTGATGCGGTTTT |
| | GGCAGTACACCAATGGGCGTGGATAGCGGTTT |
| | GACTCACGGGGATTTCCAAGTCTCCACCCCATTGACGTCAATGGGAG |
| | TTTGTTTTGGCACCAAAATCAACGGGACTTTCC |
| | AAAATGTCGTAATAACCCCGCCCCGTTGACGCAAATGGGCGGTAGGC |
| | GTGTACGGTGGGAGGTCTATATAAGCAGAGCTC |
| | GTTTAGTGAACCGTCAGATCACTAGAAGCTTTATTGCGGTAGTTTATC |
| | ACAGTTAAATTGCTAACGCAGTCAGTGGGCCT |
| | CGGCGGCCAAGCTAGGCAATCCGGTACTGTTGGTAAAGCCACCATGG |
| | TCTTCACACTCGAAGATTTCGTTGGGGACTGGC |
| | GACAGACAGCCGGCTACAACCTGGACCAAGTCCTCGAGCAGGGAGG |
| | TGTGTCCAGTTTGTTTCAGAATCTCGGGGTGTCC |
| | GTAACTCCGATCCAAAGGATTGTCCTGAGCGGTGAAAATGGGCTGAA |
| | GATCGACATCCATGTCATCATCCCGTATGAAGG |
| | TCTGAGCGGCGACCAAATGGGCCAGATCGAAAAAATTTTTAAGGTGG |
| | TGTACCCTGTGGATGATCATCACTTTAAGGTGA |
| | TCCTGCACTATGGCACACTGGTAATCGACGGGGTTACGCCGAACATG |
| | ATCGACTATTTCGGACGGCCGTATGAAGGCATC |
| | GCCGTGTTCGACGGCAAAAAGATCACTGTAACAGGGACCCTGTGGAA |
| | CGGCAACAAAATTATCGACGAGCGCCTGATCAA |
| | CCCCGACGGCTCCCTGCTGTTCCGAGTAACCATCAACGGAGTGACCG |
| | GCTGGCGGCTGTGCGAACGCATTCTGGCGAATT |
| | CTCACGGCTTTCCGCCTGAGGTTGAAGAGCAAGCCGCCGGTACATTG |
| | CCTATGTCCTGCGCACAAGAAAGCGGTATGGAC |
| | CGGCACCCAGCCGCTTGTGCTTCAGCTCGCATCAACGTCTAAGGCCG |
| | CGACTCTAGAGTCGGGGCGGCCGGCCGCTTCGA |
| | GCAGACATGATAAGATACATTGATGAGTTTGGACAAACCACAACTAGA |
| | ATGCAGTGAAAAAAATGCTTTATTTGTGAAAT |
| | TTGTGATGCTATTGCTTTATTTGTAACCATTATAAGCTGCAATAAACAA |
| | GTTAACAACAACAATTGCATTCATTTTATGT |
| | TTCAGGTTCAGGGGGAGGTGTGGGAGGTTTTTTAAAGCAAGTAAAAC |
| | CTCTACAAATGTGGTAAAATCGATAAGGATCCG |
| | TTTGCGTATTGGGCGCTCTTCCGCTGATCTGGTACCGTTTAAACCTGG |
| | GCCTCATGGGCCTTCCTTTCACTGCCCGCTTT |
| | CCAGTCGGGAAACCTGTCGTGCCAGCTGCATTAACATGGTCATAGCT |
| | GTTTCCTTGCGTATTGGGCGCTCTCCGCTTCCT |
| | CGCTCACTGACTCGCTGCGCTCGGTCGTTCGGGTAAAGCCTGGGGT |
| | GCCTAATGAGCAAAAGGCCAGCAAAAGGCCAGGA |
| | ACCGTAAAAAGGCCGCGTTGCTGGCGTTTTTCCATAGGCTCCGCCCC |
| | CCTGACGAGCATCACAAAAATCGACGCTCAAGT |
| | CAGAGGTGGCGAAACCCGACAGGACTATAAAGATACCAGGCGTTTCC |
| | CCCTGGAAGCTCCCTCGTGCGCTCTCCTGTTCC |
| | GACCCTGCCGCTTACCGGATACCTGTCCGCCTTTCTCCCTTCGGGAA |
| | GCGTGGCGCTTTCTCATAGCTCACGCTGTAGGT |
| | ATCTCAGTTCGGTGTAGGTCGTTCGCTCCAAGCTGGGCTGTGTGCAC |
| | GAACCCCCCGTTCAGCCCGACCGCTGCGCCTTA |
| | TCCGGTAACTATCGTCTTGAGTCCAACCCGGTAAGACACGACTTATCG |
| | CCACTGGCAGCAGCCACTGGTAACAGGATTAG |
| | CAGAGCGAGGTATGTAGGCGGTGCTACAGAGTTCTTGAAGTGGTGGC |
| | CTAACTACGGCTACACTAGAAGAACAGTATTTG |
| | GTATCTGCGCTCTGCTGAAGCCAGTTACCTTCGGAAAAAGAGTTGGTA |
| | GCTCTTGATCCGGCAAACAAACCACCGCTGGT |
| | AGCGGTGGTTTTTTTGTTTGCAAGCAGCAGATTACGCGCAGAAAAAAA |
| | GGATCTCAAGAAGATCCTTTGATCTTTTCTAC |
| | GGGGTCTGACGCTCAGTGGAACGAAAACTCACGTTAAGGGATTTTGG |
| | TCATGAGATTATCAAAAAGGATCTTCACCTAGA |
| | TCCTTTTAAATTAAAAATGAAGTTTTAAATCAATCTAAAGTATATATGAG |
| | TAAACTTGGTCTGACAGTTATTAGAAAAAT |
| | TCATCCAGCAGACGATAAAACGCAATACGCTGGCTATCCGGTGCCGC |
| | AATGCCATACAGCACCAGAAAACGATCCGCCCA |
| | TTCGCCGCCCAGTTCTTCCGCAATATCACGGGTGGCCAGCGCAATAT |
| | CCTGATAACGATCCGCCACGCCCAGACGGCCGC |
| | AATCAATAAAGCCGCTAAAACGGCCATTTTCCACCATAATGTTCGGCA |
| | GGCACGCATCACCATGGGTCACCACCAGATCT |
| | TCGCCATCCGGCATGCTCGCTTTCAGACGCGCAAACAGCTCTGCCGG |
| | TGCCAGGCCCTGATGTTCTTCATCCAGATCATC |
| | CTGATCCACCAGGCCCGCTTCCATACGGGTACGCGCACGTTCAATAC |
| | GATGTTTCGCCTGATGATCAAACGGACAGGTCG |
| | CCGGGTCCAGGGTATGCAGACGACGCATGGCATCCGCCATAATGCTC |
| | ACTTTTTCTGCCGGCGCCAGATGGCTAGACAGC |
| | AGATCCTGACCCGGCACTTCGCCCAGCAGCAGCCAATCACGGCCCG |
| | CTTCGGTCACCACATCCAGCACCGCCGCACACGG |
| | AACACCGGTGGTGGCCAGCCAGCTCAGACGCGCCGCTTCATCCTGC |
| | AGCTCGTTCAGCGCACCGCTCAGATCGGTTTTCA |
| | CAAACAGCACCGGACGACCCTGCGCGCTCAGACGAAACACCGCCGC |
| | ATCAGAGCAGCCAATGGTCTGCTGCGCCCAATCA |
| | TAGCCAAACAGACGTTCCACCCACGCTGCCGGGCTACCCGCATGCAG |
| | GCCATCCTGTTCAATCATACTCTTCCTTTTTCA |
| | ATATTATTGAAGCATTTATCAGGGTTATTGTCTCATGAGCGGATACATA |
| | TTTGAATGTATTTAGAAAAATAAACAAATAG |
| | GGGTTCCGCGCACATTTCCCCGAAAAGTGCCAC (SEQ ID NO: 2) |
| Complete | CTAAATTGTAAGCGTTAATATTTTGTTAAAATTCGCGTTAAATTTTTGTT |
| plasmid | AAATCAGCTCATTTTTTAACCAATAGGCCG |
| sequence of | AAATCGGCAAAATCCCTTATAAATCAAAAGAATAGACCGAGATAGGGT |
| pMK- | TGAGTGGCCGCTACAGGGCGCTCCCATTCGCC |
| RQ/NL-pos- | ATTCAGGCTGCGCAACTGTTGGGAAGGGCGTTTCGGTGCGGGCCTCT |
| ctrl-NHEJ | TCGCTATTACGCCAGCTGGCGAAAGGGGGATGT |
| | GCTGCAAGGCGATTAAGTTGGGTAACGCCAGGGTTTTCCCAGTCACG |
| | ACGTTGTAAAACGACGGCCAGTGAGCGCGACGT |
| | AATACGACTCACTATAGGGCGAATTGAAGGAAGGCCGTCAAGGCCGC |
| | ATGTTTAAACGCGGCCGCGGCCTAACTGGCCTC |
| | AATATTGGCCATTAGCCATATTATTCATTGGTTATATAGCATAAATCAAT |
| | ATTGGCTATTGGCCATTGCATACGTTGTAT |
| | CTATATCATAATATGTACATTTATATTGGCTCATGTCCAATATGACCGC |
| | CATGTTGGCATTGATTATTGACTAGTTATTA |
| | ATAGTAATCAATTACGGGGTCATTAGTTCATAGCCCATATATGGAGTTC |
| | CGCGTTACATAACTTACGGTAAATGGCCCGC |
| | CTGGCTGACCGCCCAACGACCCCCGCCCATTGACGTCAATAATGACG |
| | TATGTTCCCATAGTAACGCCAATAGGGACTTTC |
| | CATTGACGTCAATGGGTGGAGTATTTACGGTAAACTGCCCACTTGGCA |
| | GTACATCAAGTGTATCATATGCCAAGTCCGCC |
| | CCCTATTGACGTCAATGACGGTAAATGGCCCGCCTGGCATTATGCCC |
| | AGTACATGACCTTACGGGACTTTCCTACTTGGC |
| | AGTACATCTACGTATTAGTCATCGCTATTACCATGGTGATGCGGTTTT |
| | GGCAGTACACCAATGGGCGTGGATAGCGGTTT |
| | GACTCACGGGGATTTCCAAGTCTCCACCCCATTGACGTCAATGGGAG |
| | TTTGTTTTGGCACCAAAATCAACGGGACTTTCC |
| | AAAATGTCGTAATAACCCCGCCCCGTTGACGCAAATGGGCGGTAGGC |
| | GTGTACGGTGGGAGGTCTATATAAGCAGAGCTC |
| | GTTTAGTGAACCGTCAGATCACTAGAAGCTTTATTGCGGTAGTTTATC |
| | ACAGTTAAATTGCTAACGCAGTCAGTGGGCCT |
| | CGGCCGGCTACAACCTGGACCAAGTCCTCGAGCAGGGAGGTGTGTC |
| | CAGTTTGTTTCAGAATCTCGGGGTGTCCGTAACT |
| | CCGATCCAAAGGATTGTCCTGAGCGGTGAAAATGGGCTGAAGATCGA |
| | CATCCATGTCATCATCCCGTATGAAGGTCTGAG |
| | CGGCGACCAAATGGGCCAGATCGAAAAAATTTTTAAGGTGGTGTACC |
| | CTGTGGATGATCATCACTTTAAGGTGATCCTGC |
| | ACTATGGCACACTGGTAATCGACGGGGTTACGCCGAACATGATCGAC |
| | TATTTCGGACGGCCGTATGAAGGCATCGCCGTG |
| | TTCGACGGCAAAAAGATCACTGTAACAGGGACCCTGTGGAACGGCAA |
| | CAAAATTATCGACGAGCGCCTGATCAACCCCGA |
| | CGGCTCCCTGCTGTTCCGAGTAACCATCAACGGAGTGACCGGCTGGC |
| | GGCTGTGCGAACGCATTCTGGCGAATTCTCACG |
| | GCTTTCCGCCTGAGGTTGAAGAGCAAGCCGCCGGTACATTGCCTATG |
| | TCCTGCGCACAAGAAAGCGGTATGGACCGGCAC |
| | CCAGCCGCTTGTGCTTCAGCTCGCATCAACGTCTAAGGCCGCGACTC |
| | TAGAGTCGGGGCGGCCGGCCGCTTCGAGCAGAC |
| | ATGATAAGATACATTGATGAGTTTGGACAAACCACAACTAGAATGCAG |
| | TGAAAAAAATGCTTTATTTGTGAAATTTGTGA |
| | TGCTATTGCTTTATTTGTAACCATTATAAGCTGCAATAAACAAGTTAAC |
| | AACAACAATTGCATTCATTTTATGTTTCAGG |
| | TTCAGGGGGAGGTGTGGGAGGTTTTTTAAAGCAAGTAAAACCTCTACA |
| | AATGTGGTAAAATCGATAAGGATCCGTTTGCG |
| | TATTGGGCGCTCTTCCGCTGATCTGGTACCGTTTAAACCTGGGCCTCA |
| | TGGGCCTTCCTTTCACTGCCCGCTTTCCAGTC |
| | GGGAAACCTGTCGTGCCAGCTGCATTAACATGGTCATAGCTGTTTCCT |
| | TGCGTATTGGGCGCTCTCCGCTTCCTCGCTCA |
| | CTGACTCGCTGCGCTCGGTCGTTCGGGTAAAGCCTGGGGTGCCTAAT |
| | GAGCAAAAGGCCAGCAAAAGGCCAGGAACCGTA |
| | AAAAGGCCGCGTTGCTGGCGTTTTTCCATAGGCTCCGCCCCCCTGAC |
| | GAGCATCACAAAAATCGACGCTCAAGTCAGAGG |
| | TGGCGAAACCCGACAGGACTATAAAGATACCAGGCGTTTCCCCCTGG |
| | AAGCTCCCTCGTGCGCTCTCCTGTTCCGACCCT |
| | GCCGCTTACCGGATACCTGTCCGCCTTTCTCCCTTCGGGAAGCGTGG |
| | CGCTTTCTCATAGCTCACGCTGTAGGTATCTCA |
| | GTTCGGTGTAGGTCGTTCGCTCCAAGCTGGGCTGTGTGCACGAACCC |
| | CCCGTTCAGCCCGACCGCTGCGCCTTATCCGGT |
| | AACTATCGTCTTGAGTCCAACCCGGTAAGACACGACTTATCGCCACTG |
| | GCAGCAGCCACTGGTAACAGGATTAGCAGAGC |
| | GAGGTATGTAGGCGGTGCTACAGAGTTCTTGAAGTGGTGGCCTAACT |
| | ACGGCTACACTAGAAGAACAGTATTTGGTATCT |
| | GCGCTCTGCTGAAGCCAGTTACCTTCGGAAAAAGAGTTGGTAGCTCTT |
| | GATCCGGCAAACAAACCACCGCTGGTAGCGGT |
| | GGTTTTTTTGTTTGCAAGCAGCAGATTACGCGCAGAAAAAAAGGATCT |
| | CAAGAAGATCCTTTGATCTTTTCTACGGGGTC |
| | TGACGCTCAGTGGAACGAAAACTCACGTTAAGGGATTTTGGTCATGAG |
| | ATTATCAAAAAGGATCTTCACCTAGATCCTTT |
| | TAAATTAAAAATGAAGTTTTAAATCAATCTAAAGTATATATGAGTAAACT |
| | TGGTCTGACAGTTATTAGAAAAATTCATCC |
| | AGCAGACGATAAAACGCAATACGCTGGCTATCCGGTGCCGCAATGCC |
| | ATACAGCACCAGAAAACGATCCGCCCATTCGCC |
| | GCCCAGTTCTTCCGCAATATCACGGGTGGCCAGCGCAATATCCTGAT |
| | AACGATCCGCCACGCCCAGACGGCCGCAATCAA |
| | TAAAGCCGCTAAAACGGCCATTTTCCACCATAATGTTCGGCAGGCACG |
| | CATCACCATGGGTCACCACCAGATCTTCGCCA |
| | TCCGGCATGCTCGCTTTCAGACGCGCAAACAGCTCTGCCGGTGCCAG |
| | GCCCTGATGTTCTTCATCCAGATCATCCTGATC |
| | CACCAGGCCCGCTTCCATACGGGTACGCGCACGTTCAATACGATGTT |
| | TCGCCTGATGATCAAACGGACAGGTCGCCGGGT |
| | CCAGGGTATGCAGACGACGCATGGCATCCGCCATAATGCTCACTTTTT |
| | CTGCCGGCGCCAGATGGCTAGACAGCAGATCC |
| | TGACCCGGCACTTCGCCCAGCAGCAGCCAATCACGGCCCGCTTCGG |
| | TCACCACATCCAGCACCGCCGCACACGGAACACC |
| | GGTGGTGGCCAGCCAGCTCAGACGCGCCGCTTCATCCTGCAGCTCG |
| | TTCAGCGCACCGCTCAGATCGGTTTTCACAAACA |
| | GCACCGGACGACCCTGCGCGCTCAGACGAAACACCGCCGCATCAGA |
| | GCAGCCAATGGTCTGCTGCGCCCAATCATAGCCA |
| | AACAGACGTTCCACCCACGCTGCCGGGCTACCCGCATGCAGGCCATC |
| | CTGTTCAATCATACTCTTCCTTTTTCAATATTA |
| | TTGAAGCATTTATCAGGGTTATTGTCTCATGAGCGGATACATATTTGAA |
| | TGTATTTAGAAAAATAAACAAATAGGGGTTC |
| | CGCGCACATTTCCCCGAAAAGTGCCAC (SEQ ID NO: 3) |
| Complete | TCGCGCGTTTCGGTGATGACGGTGAAAACCTCTGACACATGCAGCTC |
| plasmid | CCGGAGACGGTCACAGCTTGTCTGTAAGCGGAT |
| sequence of | GCCGGGAGCAGACAAGCCCGTCAGGGCGCGTCAGCGGGTGTTGGC |
| pUC57/Eco | GGGTGTCGGGGCTGGCTTAACTATGCGGCATCAGA |
| RV NL | GCAGATTGTACTGAGAGTGCACCATATGCGGTGTGAAATACCGCACA |
| InternalNHE | GATGCGTAAGGAGAAAATACCGCATCAGGCGCC |
| J rep - no | ATTCGCCATTCAGGCTGCGCAACTGTTGGGAAGGGCGATCGGTGCG |
| PEST. The | GGCCTCTTCGCTATTACGCCAGCTGGCGAAAGGG |
| core | GGATGTGCTGCAAGGCGATTAAGTTGGGTAACGCCAGGGTTTTCCCA |
| substrate | GTCACGACGTTGTAAAACGACGGCCAGTGAATT |
| fragment | GACGCGTATTGGGATATCCATGTCATCATCCCGTATGAAGGTCTGAGC |
| excised by | GGCGACCAAATGGGCCAGATCGAAAAAATTTT |
| EcoRV | TAAGGTGGTGTACCCTGTGGATGATCATCACTTTAAGGTGATCCTGCA |
| digestion is | CTATGGCACACTGGTAATCGACGGGGTTACGC |
| in italics. | CGAACATGATCGACTATTTCGGACGGCCGTATGAAGGCATCGCCGTG |
| Enzyme | TTCGACGGCAAAAAGATCACTGTAACAGGGACC |
| recognition | CTGTGGAACGGCAACAAAATTATCGACGAGCGCCTGATCAACCCCGA |
| sites are | CGGCTCCCTGCTGTTCCGAGTAACCATCAACGG |
| underlined. | AGTGACCGGCTGGCGGCTGTGCGAACGCATTCTGGCG |
| | TAAGGCCGCGACTCTAGAGTCGGGGCGGCCGGCCGCTTCGAGCAGA |
| | CATGATAAGATACATTGATGAGTTTGGACAAACC |
| | ACAACTAGAATGCAGTGAAAAAAATGCTTTATTTGTGAAATTTGTGATG |
| | CTATTGCTTTATTTGTAACCATTATAAGCTG |
| | CAATAAACAAGTTAACAACAACAATTGCATTCATTTTATGTTTCAGGTT |
| | CAGGGGGAGGTGTGGGAGGTTTTTTAAAGCA |
| | AGTAAAACCTCTACAAATGTGGTAAAATCGATAAGGATCCGTTTGCGT |
| | ATTGGGCGCTCTTCCGCTGATCTGGCCTAACT |
| | GGCCTCAATATTGGCCATTAGCCATATTATTCATTGGTTATATAGCATA |
| | AATCAATATTGGCTATTGGCCATTGCATACG |
| | TTGTATCTATATCATAATATGTACATTTATATTGGCTCATGTCCAATATG |
| | ACCGCCATGTTGGCATTGATTATTGACTAG |
| | TTATTAATAGTAATCAATTACGGGGTCATTAGTTCATAGCCCATATATG |
| | GAGTTCCGCGTTACATAACTTACGGTAAATG |
| | GCCCGCCTGGCTGACCGCCCAACGACCCCCGCCCATTGACGTCAATA |
| | ATGACGTATGTTCCCATAGTAACGCCAATAGGG |
| | ACTTTCCATTGACGTCAATGGGTGGAGTATTTACGGTAAACTGCCCAC |
| | TTGGCAGTACATCAAGTGTATCATATGCCAAG |
| | TCCGCCCCCTATTGACGTCAATGACGGTAAATGGCCCGCCTGGCATT |
| | ATGCCCAGTACATGACCTTACGGGACTTTCCTA |
| | CTTGGCAGTACATCTACGTATTAGTCATCGCTATTACCATGGTGATGC |
| | GGTTTTGGCAGTACACCAATGGGCGTGGATAG |
| | CGGTTTGACTCACGGGGATTTCCAAGTCTCCACCCCATTGACGTCAAT |
| | GGGAGTTTGTTTTGGCACCAAAATCAACGGGA |
| | CTTTCCAAAATGTCGTAATAACCCCGCCCCGTTGACGCAAATGGGCG |
| | GTAGGCGTGTACGGTGGGAGGTCTATATAAGCA |
| | GAGCTCGTTTAGTGAACCGTCAGATCACTAGAAGCTTTATTGCGGTAG |
| | TTTATCACAGTTAAATTGCTAACGCAGTCAGT |
| | GGGCCTCGGCGGCCAAGCTAGGCAATCCGGTACTGTTGGTAAAGCC |
| | ACCATGGTCTTCACACTCGAAGATTTCGTTGGGG |
| | ACTGGCGACAGACAGCCGGCTACAACCTGGACCAAGTCCTCGAGCA |
| | GGGAGGTGTGTCCAGTTTGTTTCAGAATCTCGGG |
| | GTCTCCGTAACTCCGATCCAAAGGATTGTCCTGAGCGGTGAAAATGG |
| | GCTGAAGATCGATATCCCAATGGCGCGCCGAGC |
| | TTGGCTCGAGCATGGTCATAGCTGTTTCCTGTGTGAAATTGTTATCCG |
| | CTCACAATTCCACACAACATACGAGCCGGAAG |
| | CATAAAGTGTAAAGCCTGGGGTGCCTAATGAGTGAGCTAACTCACATT |
| | AATTGCGTTGCGCTCACTGCCCGCTTTCCAGT |
| | CGGGAAACCTGTCGTGCCAGCTGCATTAATGAATCGGCCAACGCGCG |
| | GGGAGAGGCGGTTTGCGTATTGGGCGCTCTTCC |
| | GCTTCCTCGCTCACTGACTCGCTGCGCTCGGTCGTTCGGCTGCGGCG |
| | AGCGGTATCAGCTCACTCAAAGGCGGTAATACG |
| | GTTATCCACAGAATCAGGGGATAACGCAGGAAAGAACATGTGAGCAA |
| | AAGGCCAGCAAAAGGCCAGGAACCGTAAAAAGG |
| | CCGCGTTGCTGGCGTTTTTCCATAGGCTCCGCCCCCCTGACGAGCAT |
| | CACAAAAATCGACGCTCAAGTCAGAGGTGGCGA |
| | AACCCGACAGGACTATAAAGATACCAGGCGTTTCCCCCTGGAAGCTC |
| | CCTCGTGCGCTCTCCTGTTCCGACCCTGCCGCT |
| | TACCGGATACCTGTCCGCCTTTCTCCCTTCGGGAAGCGTGGCGCTTT |
| | CTCATAGCTCACGCTGTAGGTATCTCAGTTCGG |
| | TGTAGGTCGTTCGCTCCAAGCTGGGCTGTGTGCACGAACCCCCCGTT |
| | CAGCCCGACCGCTGCGCCTTATCCGGTAACTAT |
| | CGTCTTGAGTCCAACCCGGTAAGACACGACTTATCGCCACTGGCAGC |
| | AGCCACTGGTAACAGGATTAGCAGAGCGAGGTA |
| | TGTAGGCGGTGCTACAGAGTTCTTGAAGTGGTGGCCTAACTACGGCT |
| | ACACTAGAAGAACAGTATTTGGTATCTGCGCTC |
| | TGCTGAAGCCAGTTACCTTCGGAAAAAGAGTTGGTAGCTCTTGATCCG |
| | GCAAACAAACCACCGCTGGTAGCGGTGGTTTT |
| | TTTGTTTGCAAGCAGCAGATTACGCGCAGAAAAAAAGGATCTCAAGAA |
| | GATCCTTTGATCTTTTCTACGGGGTCTGACGC |
| | TCAGTGGAACGAAAACTCACGTTAAGGGATTTTGGTCATGAGATTATC |
| | AAAAAGGATCTTCACCTAGATCCTTTTAAATT |
| | AAAAATGAAGTTTTAAATCAATCTAAAGTATATATGAGTAAACTTGGTC |
| | TGACAGTTAGAAAAACTCATCGAGCATCAAA |
| | TGAAACTGCAATTTATTCATATCAGGATTATCAATACCATATTTTTGAAA |
| | AAGCCGTTTCTGTAATGAAGGAGAAAACTC |
| | ACCGAGGCAGTTCCATAGGATGGCAAGATCCTGGTATCGGTCTGCGA |
| | TTCCGACTCGTCCAACATCAATACAACCTATTA |
| | ATTTCCCCTCGTCAAAAATAAGGTTATCAAGTGAGAAATCACCATGAG |
| | TGACGACTGAATCCGGTGAGAATGGCAAAAGT |
| | TTATGCATTTCTTTCCAGACTTGTTCAACAGGCCAGCCATTACGCTCG |
| | TCATCAAAATCACTCGCATCAACCAAACCGTT |
| | ATTCATTCGTGATTGCGCCTGAGCGAGACGAAATACGCGATCGCTGTT |
| | AAAAGGACAATTACAAACAGGAATCGAATGCA |
| | ACCGGCGCAGGAACACTGCCAGCGCATCAACAATATTTTCACCTGAAT |
| | CAGGATATTCTTCTAATACCTGGAATGCTGTT |
| | TTCCCAGGGATCGCAGTGGTGAGTAACCATGCATCATCAGGAGTACG |
| | GATAAAATGCTTGATGGTCGGAAGAGGCATAAA |
| | TTCCGTCAGCCAGTTTAGTCTGACCATCTCATCTGTAACATCATTGGC |
| | AACGCTACCTTTGCCATGTTTCAGAAACAACT |
| | CTGGCGCATCGGGCTTCCCATACAATCGATAGATTGTCGCACCTGATT |
| | GCCCGACATTATCGCGAGCCCATTTATACCCA |
| | TATAAATCAGCATCCATGTTGGAATTTAATCGCGGCCTAGAGCAAGAC |
| | GTTTCCCGTTGAATATGGCTCATACTCTTCCT |
| | TTTTCAATATTATTGAAGCATTTATCAGGGTTATTGTCTCATGAGCGGA |
| | TACATATTTGAATGTATTTAGAAAAATAAAC |
| | AAATAGGGGTTCCGCGCACATTTCCCCGAAAAGTGCCACCTGACGTC |
| | TAAGAAACCATTATTATCATGACATTAACCTAT |
| | AAAAATAGGCGTATCACGAGGCCCTTTCGTC (SEQ ID NO: 4) |

Two nucleic acid caps were generated by annealing pairs of oligonucleotides (Table 2) by controlled cooling in annealing buffer (20 mM Tris-HCl, pH7.5, 50 mM NaCl) in a PCR thermocycler.

TABLE 2
Primers for Caps

| Oligo | |
|---|---|
| Name | Sequence |
| Left | 5′[Phos] |
| cap | TCGAGGACTTGGTCCAGGTTGTAGCCGGCTGTCTGTCGCC |
| long | AGTCCCCAACGAAATCTTCGAGTGTGAAGACAT |
| | (SEQ ID NO: 5) |
| Left | 5′[Phos] |
| cap | GCCGGCTACAACCTGGACCAAGTCC |
| short | (SEQ ID NO: 6) |
| Right | 5′[Phos] |
| cap | AGCTTTATTGCGGTAGTTTATCACAGTTAAATTGCTAACG |
| long | CAGTCAGTGGGCCTCGGCGGCCAAGCTAGGCAATCCGGTA |
| | CTGTTGGTAAAGCCACCATGG |
| | (SEQ ID NO: 7) |
| Right | 5′[Phos] |
| cap | CGAGGCCCACTGACTGCGTTAGCAATTTAACTGTGAT |
| short | AAACTACCGCAATAA |
| | (SEQ ID NO: 8) |

Each cap comprised two functional ends: a 4-nt “sticky end” suitable for specific ligation to a complementary end of the core substrate fragment (Left=XhoI, Right=HindIII) and a 45-nt 3′-ssDNA overhang comprising a terminal 4-nt microhomology to the other cap.

The caps were ligated onto the substrate fragment in a 6:6:1 ratio (left cap: right cap: core) at 16° C. overnight using T4 DNA ligase (NEB). The ligations were purified using a PCR purification kit (Qiagen) and separated from unligated excess caps by a further round of agarose gel electrophoresis and gel extraction.

Control plasmids pMK-RQ/NL-pos-ctrl-MMEJ and pMK-RQ/NL-pos-ctrl-NHEJ, mimicking the products of MMEJ- and NHEJ-mediated repair, of the MMEJ reporter, were generated by gene synthesis (GeneArt).

The generation of an NHEJ substrate is outlined in FIG. 8. The vector denoted as “pUC57/EcoRV NL InternalNHEJ rep—no PEST”, comprising a CMV promoter downstream of the C-terminal region of the NanoLuciferase gene and SV40 poly A terminator, and upstream of the N-terminal region of NanoLuciferase was generated by gene synthesis (GeneWiz) (Table 1). The sequences were derived, and rearranged, from pNL3.2CMV (Promega) with the addition the incorporation of silent nucleotide substitutions introducing an EcoRV restriction site into the NanoLuciferase coding region.

A region of the vector was excised by restriction digest with EcoRV. The blunt end fragment constituting the reporter substrate was separated from the vector backbone by agarose gel electrophoresis and purified by gel extraction (Qiagen).

Cell Culture

HEK293 cells (ATCC) were cultured in MEM Eagle media (PAN-Biotech) supplemented with 10% foetal bovine serum (FBS) (PAN-Biotech) under normal growth conditions (37° C., 5% CO₂), and passaged at 80% confluency. Wild-type, XRCC4^{-/-}, and XLF^{-/-}HCT116 cells (Horizon Discovery) were cultured in RPMI-1640 media (PAN-Biotech) supplemented with 10% foetal bovine serum (FBS) (PAN-Biotech) under normal growth conditions (37° C., 5% CO₂), and passaged at 80% confluency.

Cellular MMEJ/NHEJ Assays

HEK293 cells were harvested by trypsinisation, washed with PBS, resuspended in fresh media, and counted. Cells were centrifuged at 400 g for five minutes, and resuspended in supplemented SF nucleofection solution (Lonza) containing the NanoLuciferase DNA substrate and FireFly luciferase plasmid (Promega) at a ratio of 20 μL SF: 200 ng NanoLuciferase substrate: 400 ng FireFly plasmid: 200,000 cells. Cells were transferred to a cuvette, electroporated using the program CM-130 on the 4D nucleofector X unit (Lonza) and recovered into fresh media to a final density of 125,000 cells/mL. 10,000 cells (80 μL of suspension) were seeded per well in a white 96-well microplate (Costar 3610) and incubated for 24 hours at 37° C.

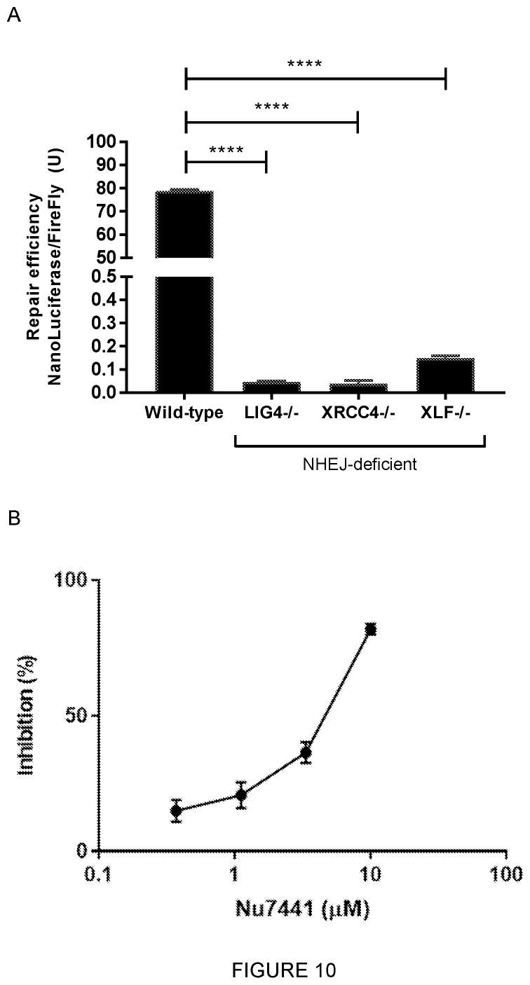

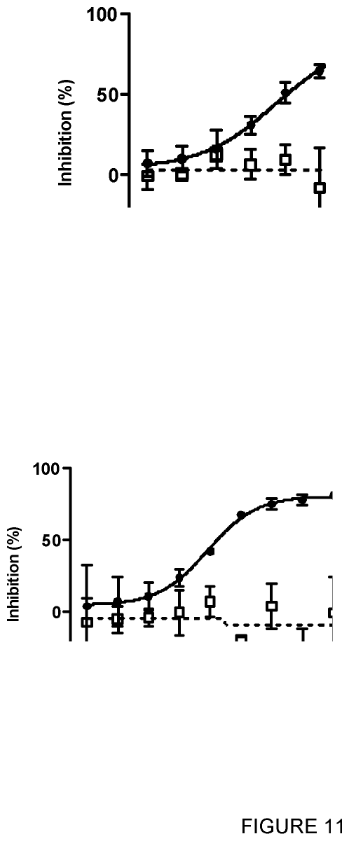

To assess titratable inhibition of MMEJ (FIG. 6), compounds were dispensed using the Tecan D300e digital dispenser to generate a 12-point dose response curve (top concentration 12 μM, dilution factor 3), with a backfilling step included to equalise the final DMSO concentration to 0.1%. Cells were transfected with the MMEJ substrate and plated to wells containing compound. To assess titratable inhibition of NHEJ (FIG. 10B), cells were preincubated with NU7441, an inhibitor of DNA-PKcs for 2 hours, prior to transfection with the NHEJ reporter. Cells were then plated to wells containing compound. Firefly and NanoLuciferase levels were detected using the Nano-Glo® Dual-Luciferase®

Reporter Assay system (Promega) as per the manufacturer's instructions, and luminescence was measured with a Clariostar plate reader (BMG Labtech), using the manufacturer's protocols ‘FireFly’ and ‘NanoLuciferase’. In each well the NanoLuciferase signal was normalised to the Firefly signal, which served as a measure of both cell density and transfection efficiency.

Cellular NHEJ Assays

HCT116 cells (Wild-type and NHEJ-deficient) were harvested by trypsinisation, washed with media, resuspended in fresh media, and counted. Cells were centrifuged at 400 g for five minutes and resuspended in supplemented SE nucleofection solution (Lonza) containing the

NanoLuciferase DNA substrate and FireFly luciferase plasmid (Promega) at a ratio of 20 μL SE: 1 μg NanoLuciferase substrate: 400 ng FireFly plasmid: 200,000 cells. Cells were transferred to a cuvette, electroporated using the programme EN-113 on the 4D nucleofector X unit (Lonza) and recovered into fresh media to a final density of 250,000 cells/mL. 20,000 cells (80 μL of suspension) were seeded per well in a white 96-well microplate (Costar 3610) and incubated for 24 hours at 37° C.

Firefly and NanoLuciferase levels were detected using the Nano-Glo® Dual-Luciferase® Reporter Assay system (Promega) as per the manufacturer's instructions, and luminescence was measured with a Clariostar plate reader (BMG Labtech), using the manufacturer's protocols ‘FireFly’ and ‘NanoLuciferase’. In each well the NanoLuciferase signal was normalised to the Firefly signal, which served as a measure of both cell density and transfection efficiency. The results of this study are shown in FIG. 10 where it can be seen that the repair efficiency of the reporter is significantly reduced in NHEJ-deficient genetic backgrounds (LIG4^{-/-}, XRCC4^{-/-}, and XLF^{-/-}), supporting a direct tole for NHEJ machinery in its repair.

gDNA Isolation, Polymerase Chain Reaction, and Gel Electrophoresis

Cell pellets were washed twice with 1 ml PBS and resuspended in 200 μl PBS. Genomic DNA was isolated using the QIAamp DNA Mini Kit (Qiagen) as per the manufacturer's instructions, with an elution volume of 35 μL AE buffer. Samples were diluted to 20 ng/μL in EB buffer (Qiagen), and the polymerase chain reaction was carried out using the KOD Hot Start Polymerase kit (Merck), primers ‘CMV forward’ (CGCAAATGGGCGGTAGGCGTG; SEQ ID NO: 9) and ‘SV40 reverse’ (GTGGTTTGTCCAAACTCATC; SEQ ID NO: 10), and 100 ng gDNA template per reaction. The PCR reaction was carried out in an Eppendorf Mastercycler Nexus thermocycler using the following programme: 95° C. for 2 minutes, 35 cycles of [95° C. for 20 seconds, 59° C. for 10 seconds, 70° C. for 15 seconds], 70° C. for 1 minute. For controls to generate the expected MMEJ and NHEJ repair products, PCR was performed using pMK-RQ/NL-pos-ctrl-MMEJ and pMK-RQ/NL-pos-ctrl-NHEJ plasmid templates, respectively.

Samples were resolved on a 4-20% polyacrylamide TBE gel (Life Technologies). Gels were stained with SYBRSafe DNA gel stain (Invitrogen) and imaged on an Amersham Gel Doc. Bands were quantified using the software ImageQuant (GE Healthcare).

Cellular MMEJ Assays (Compound A and D, FIG. 11)

HEK293 cells were washed with PBS, harvested by trypsinisation, resuspended in fresh complete medium, and counted. Cells were centrifuged at 300 g for three minutes and resuspended in supplemented SF nucleofection solution (Lonza) containing the NanoLuciferase DNA substrate (lacking a PEST domain) and FireFly luciferase plasmid (Promega) at a ratio of 100 μL SF: 2500 ng NanoLuciferase substrate: 1000 ng FireFly plasmid: 1,600,000 cells. Cells were transferred to a cuvette, electroporated using the program CM-130 on the 4D nucleofector X unit (Lonza) and recovered into fresh media to a final density of 190,000 cells/mL. Approximately 15,000 cells (80 μL of suspension) were seeded per well in a white 96-well microplate (Costar 3610), in which compounds had been dispensed using the Tecan D300e digital dispenser to generate a 9-point dose response curve (top concentration 12 μM, dilution factor 3), with a backfilling step included to equalise the final DMSO concentration to 0.25% (v/v). Cells were incubated for 24 hours at 37° C.

Generation of NanoLuc (noPEST) Ramsden MMEJ core plasmid

To generate a version of the MMEJ substrate lacking the PEST domain C-terminal to Nanoluciferase (FIG. 11), an XmnI/XbaI fragment in plasmid pMK-RQ-NLcoreMMEJreporter was replaced by a duplex DNA molecule generated from annealed oligonucleotides (Forward Oligonucleotide: 5′[Phos]CATTCTGGCGTAAGGCCGCGACT (SEQ ID NO: 11); Reverse Oligonucleotide: 5′[Phos]CTAGAGTCGCGGCCTTACGCCAGAATG (SEQ ID NO: 12)). This substrate was then generated as described previously.

DISCUSSION

In this study, the inventors have described linear extrachromosomal nucleic acid substrates that can quantify the cellular capacity of MMEJ and NHEJ repair pathways. These reporters can be used in multiple contexts such as determining the integrity of DNA repair in cells (e.g. classification as proficient or deficient) or screening for small molecule modulators of these pathways.

The cellular NHEJ and MMEJ reporters described herein have specific advantages over existing assays. The use of NanoLuciferase as the reporter in the ORF makes the assay particularly suitable for experimental contexts reliant on reproducibility, robustness, quantification and increased throughput such as small molecule screening in a high-throughput format and the generation of multipoint dose responses to determine cellular EC50s.

Key advantages:
1. Produces a robust and quantifiable luminescent signal detectable by a plate reader;
2. Format is easily scalable to high throughput;
3. Sensitive to small molecule inhibition; and
4. Modulation of repair results in a titratable signal.

The substrate described explicitly reports on the use of the terminal microhomology of 3′ overhangs to stimulate MMEJ and generate a full length, functional NanoLuciferase. It cannot detect alternative repair mechanisms on the substrate that may include NHEJ, or an MMEJ-mediated repair event using a short microhomology within the overhangs that circumvents the initiator methionine.

Furthermore, these extrachromosomal reporters have advantages over chromosomally-integrated reporters that are conventionally used to measure DNA repair. The advantage of these systems is that they utilise stable cell lines encoding these reporters that can be easily generated and maintained, and enable DNA repair occurs within a physiological context, such as chromatinised DNA. These systems can also be performed in any transfectable cell line within short time frames (<24 h), thereby capturing repair events without extended compound treatments. In addition, the ability to generate repair-ready engineered nucleic acid substrates containing diverse lesions and functions makes these reporters adaptable for an array of DNA repair pathways.

1. A non-homologous end-joining (NHEJ) or a microhomology-mediated end joining (MMEJ) detection construct comprising:
(a) an open-reading frame (ORF) which encodes a reporter;
(b) a terminator sequence; and
(c) a promoter downstream of said terminator sequence,
wherein a portion of said reporter is downstream of said promoter and the remaining portion of said reporter is upstream of said terminator sequence.

2. The NHEJ or MMEJ detection construct according to claim 1, wherein the reporter is selected from β-galactosidase (lacZ), chloramphenicol acetyltransferase (cat) and fluorescent or luminescent proteins, such as green fluorescent protein (gfp), red fluorescent protein (rfp) or NanoLuciferase, in particular a fluorescent or a luminescent protein, such as GFP or NanoLuciferase.

3. The NHEJ or MMEJ detection construct according to claim 1 or claim 2, wherein the terminator sequence is selected from a mammalian terminator sequence and includes a polyadenylation signal, such as SV40, hGH, BGH and rbGlob.

4. The NHEJ or MMEJ detection construct according to any one of claims 1 to 3, wherein the promoter is selected from a cytomegalovirus (CMV) or an SV40 promoter, such as a CMV promoter.

5. The NHEJ or MMEJ detection construct according to any one of claims 1 to 4, wherein the portion of said reporter which is downstream of said promoter comprises the first 2 to 10 nucleotides which encode said reporter, such as the first 2 to 8 nucleotides, in particular the first 2 to 7 nucleotides, especially the first 4 nucleotides, more especially the first 3 nucleotides, e.g. the start codon of said reporter.

6. The NHEJ or MMEJ detection construct according to any one of claims 1 to 5, which is a NHEJ detection construct and the portion of said reporter which is downstream of said promoter and the remaining portion of said reporter which is upstream of said terminator sequence are both blunt ended.

7. The NHEJ or MMEJ detection construct according to any one of claims 1 to 5, which is an MMEJ detection construct and the portion of said reporter which is downstream of said promoter is single stranded.

8. The MMEJ detection construct according to claim 7, wherein said single stranded portion of said reporter which is downstream of said promoter comprises the first 2 to 10 nucleotides which encode said reporter, such as the first 2 to 8 nucleotides, in particular the first 2 to 7 nucleotides, especially the first 4 nucleotides (i.e. ATGX), more especially the first 3 nucleotides, e.g. the start codon of said reporter (i.e. ATG).

9. The MMEJ detection construct according to claim 7 or claim 8, wherein the terminal portion of the promoter is also single stranded.

10. The MMEJ detection construct according to any one of claims 7 to 9, wherein the portion of said reporter which is upstream of said terminator sequence is double stranded with a 3′ single stranded overhang.

11. The MMEJ detection construct according to claim 10, wherein the 3′ single stranded overhang comprises the reverse compliment of the first 100 nucleotides of said reporter, such as the first 50 nucleotides, in particular the first 45 nucleotides.

12. A non-homologous end-joining (NHEJ) or a microhomology-mediated end joining (MMEJ) detection assay which comprises the following steps:
(a) providing the NHEJ or MMEJ construct according to any one of claims 1 to 11;
(b) contacting said construct with a mediator of NHEJ or MMEJ; and
(c) detecting expression of the functional reporter.

13. The MMEJ detection assay according to claim 12, wherein the mediator of MMEJ comprises Polθ.

14. A method of screening for a modulator of NHEJ or MMEJ which comprises:
(a) providing the NHEJ or MMEJ construct according to any one of claims 1 to 11;
(b) contacting said construct with a mediator of NHEJ or MMEJ; and
(c) detecting expression of the functional reporter in both the presence and absence of a modulator of NHEJ or MMEJ,
wherein a decrease in expression relative to control is indicative of an inhibitor or antagonist of NHEJ or MMEJ and an increase in expression relative to control is indicative of an enhancer or agonist of NHEJ or MMEJ.

15. The method according to claim 14, wherein the modulator of MMEJ comprises a Polθ inhibitor.
